(19) United States
(12) Patent Application Publication
(10) Pub. No.: US 2026/0108584 A1
(43) Pub. Date: Apr. 23, 2026

(54) TREATMENT OF OBESITY-ASSOCIATED INFLAMMATORY CONDITIONS

(71) Applicant: THE UNIVERSITY OF BIRMINGHAM, Birmingham (GB)

(72) Inventors: George Edward RAINGER, Birmingham, West Midlands (GB); Helen MCGETTRICK, Birmingham, West Midlands (GB); Asif IQBAL, Birmingham, West Midlands (GB)

(73) Assignee: THE UNIVERSITY OF BIRMINGHAM, Birmingham (GB)

(21) Appl. No.: 19/152,182

(22) PCT Filed: Jan. 30, 2024
(86) PCT No.: PCT/GB2024/050244

§ 371 Date: Jul. 30, 2025

(30) Foreign Application Priority Data
Jan. 30, 2023 (GB) 2301317.0

(51) Int. Cl. A61K 38/10; A61P 3/04; A61P 3/10

(52) U.S. Cl. CPC A61K 38/10; A61P 3/04; A61P 3/10

(57) ABSTRACT

The present invention concerns a peptide comprising the amino acid sequence SVTEQGAELSNEER, or variants thereof for use in the prophylaxis or treatment of obesity-associated inflammatory conditions such as obesity-induced chronic low-grade systemic inflammation and pancreatic beta-cell damage. Methods of treatment or prophylaxis of obesity-associated inflammatory conditions comprise administering an effective amount of a peptide comprising the amino acid sequence SVTEQGAELSNEER, or variants thereof, to a patient in need thereof.

### FIELD OF THE INVENTION

The present invention concerns a peptide comprising the amino acid sequence SVTEQGAELSNEER, or variants thereof for use in the prophylaxis or treatment of obesity-associated inflammatory conditions such as obesity-induced chronic low-grade systemic inflammation and pancreatic beta-cell damage. Methods of treatment or prophylaxis of obesity-associated inflammatory conditions comprise administering an effective amount of a peptide comprising the amino acid sequence SVTEQGAELSNEER, or variants thereof, to a patient in need thereof.

### BACKGROUND OF THE INVENTION

Obesity is characterised by pathogenic changes in adipose tissues leading to low-grade systemic chronic inflammation, and aberrant accumulation of pro-inflammatory leukocytes within adipose tissues. Obese individuals have increased risk of developing co-morbidities, such as type 2 diabetes mellitus (T2DM), hepatic steatosis and cardiovascular disease, leading to higher morbidity and mortality rates compared to lean individuals. The estimated numbers of individuals who are deemed obese is raising at an alarming rate, making obesity and obesity-related diseases a major global health challenge. Despite this little is understood around how the inflammation associated with obesity drives pathology. Moreover, current clinical management often targets alternations in life-style choices (e.g., exercise, weight loss) or more extreme surgical interventions.

Obesity induces a dramatic transformation in the composition of leukocytes resident in the adipose tissue, which experiences an influx of pro-inflammatory leukocytes and detrimental changes in the homeostatic regulatory populations (Treg, NK cells, ILCs), including loss in numbers and/or the acquisition of pathogenic functions [(see Weisberg, S. P. et al., J Clin Invest 112, 1796-1808, doi: 10.1172/jci19246 (2003), and Exley, M. A., Hand, L., O'Shea, D. & Lynch, L., J Endocrinol 223, R41-48, doi: 10.1530/joe-13-0516 (2014)]. The accumulation of pro-inflammatory macrophages (so called M1 macrophages) is widely accepted to play a central role in obesity-induced pathology. However, studies suggest that the recruitment of CD8+ T-cells precedes, and potentially drives, the infiltration of M1 macrophages in murine models of obesity (see Nishimura, S. et al., Nat Med 15, 914-920, doi: 10.1038/nm. 1964 (2009)). Furthermore, intravital microscopy has been used to show augmented leukocyte adhesion both locally in visceral adipose tissue (VAT) and systemically in the cremaster muscle in obese mice when compared to non-obese controls (see Nishimura, S. et al., J Clin Invest 118, 710-721, doi: 10.1172/jci33328 (2008)).

Obese adipose tissue also exhibits dysregulation in metabolic processes involved in insulin sensitivity, leading to pancreatic beta-cell damage, insulin resistance and eventually hyperglycemia that underpins T2DM. Indeed, T2DM is characterised by increased number and size of beta cells resulting in functional insufficiency. Obesity has been reported to induce pancreatic beta cell mass expansion in obese non-diabetic patients and also mice fed on HFD for 8 weeks (see Hanley, S. C. et al., Endocrinology 151, 1462-1472, doi: 10.1210/en.2009-1277 (2010), and Tschen, S. I., Dhawan, S., Gurlo, T. & Bhushan, A. Diabetes 58, 1312-1320, doi: 10.2337/db08-1651 (2009)). In all cases, studies reported that a degree of beta cell dysfunction occurs and that this is likely to lead eventually to glucose intolerance and insulin resistance associated with T2DM.

Obesity has been reported to alter the trafficking profiles of T-cells, where memory CD4+ T-cells from HFD-fed mice preferentially migrate to non-lymphoid (i.e. peripheral tissues) in a CXCR3 dependent manner, even in chow-fed mice (see Mauro, C. et al., Cell Metab 25, 593-609, doi: 10.1016/j.cmet.2017.01.008 (2017)). Others have reported elevated numbers of senescent or senescent-associated (CD153+) T-cells within the VAT of obese mice following 16 and 18 weeks of HFD diet (see Kiran, S., Kumar, V., Murphy, E. A., Enos, R. T. & Singh, U. P. Frontiers in Immunology 12, doi: 10.3389/fimmu.2021.680944 (2021)). Senescent associated CD153+CD4+ T-cells from obese mice have been reported to drive metabolic dysregulation and inflammation within the VAT—where their adoptive transfer into non-obese mice induced insulin resistance and pro-inflammatory cytokine production in the recipient mice (see Shirakawa, K. et al., The Journal of Clinical Investigation 126, 4626-4639, doi: 10.1172/JCI88606 (2016)). Likewise, greater numbers of senescent CD8+CD57+ T-cells were detected in omental tissue of pre-diabetic or T2DM patients with BMIs in the overweight, rather than obese range, compared to normoglycemia controls (Lee, Y. H. et al., Diabetes 68, 156-162, doi: 10.2337/db17-1218 (2019)).

Whilst trafficking through secondary lymphoid organs is not routinely examined in pre-clinical models of obesity, there are a few studies describing alterations in leukocyte numbers within these tissues, which in some cases have been attributed to pathological changes in the barrier function of the lymphatics and abnormal lymph node architecture (see Weitman, E. S. et al., PLOS One 8, e70703, doi: 10.1371/journal.pone.0070703 (2013)). For example, T-cell migration into the mesenteric lymph node and dendritic cell migration into local draining lymph nodes were reduced in obese mice compared to chow-diet controls (Kim, C. S. et al., Obesity (Silver Spring) 16, 1261-1269, doi: 10.1038/oby.2008.55 (2008)). Moreover, loss of CCL21 gradients resulted in aberrant lymph node follicle organisation in obese mice, leading to a reduction in CD4+ and CD8+ T-cell numbers within the SLO (see Thomas, S. N. et al., J Immunol 189, 2181-2190, doi: 10.4049/jimmunol. 1103545 (2012)).

Adiponectin is an adipose derived adipokine that functions as insulin-sensitising hormone and anti-inflammatory cytokine. It has been shown to negatively regulate adhesion molecule expression on blood vascular endothelial cells to limit leukocyte recruitment (see Ouedraogo, R. et al., The Journal of Clinical Investigation 117, 1718-1726, doi: 10.1172/JCI29623 (2007)). The inventors have previously shown that adiponectin stimulates the release of an immunopeptide (PEPITEM) from B-cells, limiting T-cell migration into inflamed tissues (see Chimen, M. et al. Nat Med 21, 467-475, doi: 10.1038/nm.3842 (2015)). Groups have now shown the therapeutic potential of synthetic PEPITEM in murine models of immune-mediated inflammatory diseases (IMIDs). Others have reported that PEPITEM treatment reduced F4/80+ macrophage numbers in the kidneys of mice with glomerular nephritis, indicating PEPITEM can influence the trafficking of other cell types beyond T-cells as originally reported.

Recently, 14.3.35 family members (the parent protein of PEPITEM) have been postulated to influence pancreatic beta cell proliferation; significantly higher levels of proliferation have been observed in vitro when murine or human islet beta cell were cultured in the presence of a pan 14.3.3 inhibitor (see Mugabo, Y. et al., JCI Insight 7, doi: 10.1172/jci.insight. 156378 (2022)). In beta-cell specific 14.3.3 KO mice, a significant increase in pancreatic beta cells proliferation was detected but this did not alter islet mass or area (see Mugabo, Y. et al., JCI Insight 7, doi: 10.1172/jci.insight. 156378 (2022)).

Obesity has long been treated with life-style interventions (low calorie diet, and increase in exercise) followed by bariatric surgery in extreme cases. Five drugs are currently approved for use in the USA/Europe (e.g., orlistat-limits fat absorption; or lorcaserin-appetite suppressant). However, whilst providing a valuable adjunct, these drugs can often offer only moderate weight loss over that achieved with life-style interventions (reviewed in Singh, A. K. & Singh, Expert Rev Clin Pharmacol 13, 53-64, doi: 10.1080/17512433.2020.1698291 (2020)). A number of studies are investigating the benefits of therapeutic targeting of leukocyte trafficking in obesity-related inflammatory diseases, including T2DM (reviewed by Pezhman, L., Tahrani, A. & Chimen, M. Front Cell Dev Biol 9, 624184-624184, doi: 10.3389/fcell.2021.624184 (2021)). However, many of these immunomodulatory therapies only show partial reduction in disease pathogenesis.

Therefore, there is a need in the art for alternative treatments or prevention of the conditions described herein.

### SUMMARY OF THE INVENTION

Using proteomics, the inventors have identified a peptide released from B-cells after adiponectin stimulation, which they have named PEPtide Inhibitor of Trans-Endothelial Migration (PEPITEM). PEPITEM is a small peptide derived from the 14.3.3.ζδ protein by proteolytic cleavage (see Saba, J. D., nat. med. 2015, 21(5), 424-426 and Chimen, M. et al. (supra)).

The inventors have now found that peptides comprising the PEPITEM sequence, i.e. the amino acid sequence SVTEQGAELSNEER, or variants thereof are surprisingly effective in the prophylaxis or treatment of obesity-associated inflammatory conditions such as obesity-induced chronic low-grade systemic inflammation and pancreatic beta-cell damage when administered to a patient in effective amounts. Administration of such peptides to obese patients, patients suffering from type 2 diabetes mellitus, and/or at risk patient groups has the scope to treat or reduce the risk of the development of obesity-related co-morbidities.

The inventors have found that administration of PEPITEM to subjects at risk of or suffering from obesity prevents or reverses the effects of an obesogenic diet on pancreatic islet area, aberrant T-cell trafficking into visceral adipose tissue (VAT) and leukocyte mobilisation from secondary lymphoid organs (SLOs). Given that 14.3.3.ζδ (the parent protein of PEPITEM) is thought in the art to be involved for pancreatic beta-cell proliferation, but has no effect on pancreatic islet mass or area, the inventors' finding that PEPITEM is able to prevent or reverse increases in pancreatic islet area was entirely unexpected. Furthermore, the systemic differences in trafficking patterns between peripheral tissues and lymphoid tissues realised by the inventors could not be predicted from their earlier work, which focused on localised tissue-specific inflammation-driven, rather than systemic obesity-driven, leukocyte trafficking.

Viewed from a first aspect, therefore, the invention provides a peptide comprising the amino acid sequence SVTEQGAELSNEER, or variants thereof, for use in the prophylaxis or treatment of obesity-associated inflammatory conditions.

Viewed from a second aspect, the invention provides a method of prophylaxis or treatment of obesity-associated inflammatory conditions, comprising administering an effective amount of a peptide comprising the amino acid sequence SVTEQGAELSNEER, or variants thereof, to a patient in need thereof.

The skilled person is aware that any reference to an aspect of the invention may refer to any embodiment of that aspect. For the avoidance of doubt, any embodiments described herein in association with the peptide or the prophylaxis or treatment of obesity-associated inflammatory conditions of the first aspect apply mutatis mutandis to the second aspect and any further relevant aspects described herein.

### LIST OF FIGURES

FIG. 1: Prophylactic treatment with PEPITEM reduced leukocyte trafficking induced by 6 weeks of a high fat obesogenic diet.

FIG. 1(A): Schematic representation of experimental time course where wildtype male mice were prophylactically implanted with a mini pump, which continuously released 0.0822 mg/week of PEPITEM or PBS (as a control), and fed HFD for 6 weeks.

FIG. 1(B): Representative images of pancreatic islets from PBS or PEPITEM treated mice were stained with insulin producing beta cells in red and glucagon releasing alpha cells in green and imaged (scale bar=mm²). White box represents a single pancreatic islet shown at increased magnification (scale bar=μm²).

FIG. 1(C) and FIG. 1(D): Images in FIG. 1(B) were quantified for (C) the number of the islets/10000 μm² of whole area and (D) the area of islets/10000 μm² of whole area. Data points from PBS treated mice are shown as black circles on the left and data points from PEPITEM treated mice are shown as grey squares on the right.

FIG. 1(E) to FIG. 1(J): visceral adipose tissue CD3+ T-cells, (F) visceral adipose tissue CD4+ T cells, (G) visceral adipose tissue CD3+KLRG1+ T-cells, (H) peritoneal lavage CD3+ T cells, (I) peritoneal lavage CD3+KLRG1+ T cells and (J) peritoneal lavage F4/80HCD11clnt macrophages were quantified using flow cytometry analysis. Data points from PBS treated mice are shown as black circles on the left and data points from PEPITEM treated mice are shown as grey squares on the right. Absolute number of 35 immune cells were normalised to bead counts and plotted as number per g of adipose tissue and per ml of peritoneal lavage. Data are mean±SEM using n=5 mice per group from n=1 independent experiment. *=p<0.05, **=p<0.01 and *** p<0.001 by unpaired t-test compared to PBS treated.

FIG. 2: Therapeutic treatment with PEPITEM altered the patterns of leukocyte trafficking into peripheral tissues induced by 12 weeks of a high fat obesogenic diet.

FIG. 2(A): Schematic representation of experimental time course where mice were fed HFD for 12 weeks.

FIG. 2(B) to FIG. 2(K): At week 6 of the experimental time course of FIG. 2(A), mice were implanted with a mini pump to allow therapeutic continuous release 0.0822 mg/week of PEPITEM or PBS (as a control) for the last 6 weeks of the HFD. Pancreatic islets from PBS or PEPITEM treated mice were stained insulin producing beta cells in red and glucagon releasing alpha cells in green. Images were quantified for (B) the number of the islets/10000 μm2 of whole area and (C) the area of islets/10000 μm2 of whole area. (D) Visceral adipose tissue CD3+ T cells, (E) visceral adipose tissue CD4+ T cells, (F) visceral adipose tissue CD3+KLRG1+ T-cells, (G) peritoneal lavage F4/80HCD11clnt macrophages, (H) peritoneal lavage CD3+KLRG1+ T cells, (I) blood CD3+KLRG1+ T-cells, (J) blood CD19+CD43+CD93+CD23-CD21-age-associated B-cells, and (K) blood LyG6+ neutrophils were quantified using flow cytometry analysis. Data points from PBS treated mice are shown as black circles on the left and data points from PEPITEM treated mice are shown as grey squares on the right. Absolute number of immune cells were normalised to bead counts and plotted as number per g of adipose tissue and per ml of peritoneal lavage or blood. Data are mean±SEM using n=14 mice per group from n=1 independent experiment. *=p<0.05 and **=p<0.01 by unpaired t-test compared to PBS treated.

FIG. 3: Therapeutic treatment with PEPITEM altered leukocyte trafficking into secondary lymphoid tissues in obese mice. Mice were fed HFD for 12 weeks and received 0.0822 mg/week of PEPITEM or PBS as a control for the last 6 weeks of the HFD for the full duration.

FIG. 3(A) to (F): (A) Splenic CD45+ T cells, (B) splenic CD3+ T cells, (C) inguinal lymph node CD45+ T cells, (D) inguinal lymph node CD3+ T cells, (E) splenic CD19+ B cells, and (F) splenic age-associated B-cells were quantified using flow cytometry analysis. Data points from PBS treated mice are shown as black circles on the left and data points from PEPITEM treated mice are shown as grey squares on the right. Absolute number of immune cells were normalised to bead counts and plotted as number per g of tissue. Data are mean±SEM using n=14 mice per group from n=1 independent experiment. *=p<0.05 and **=p<0.01 by unpaired t-test compared to PBS treated.

FIG. 4: Flow cytometry gating strategy. Leukocytes were identified using FS/SS, doublets removed using pulse width vs FS and dead cells excluded using zombie aqua. Subsequently CD45+ leukocytes were gated from which the major subsets of cells were identified: CD3+ T-cells, CD3+CD4+ T-cells, CD3+CD8+ T-cells, CD3+KLRG1+ T-cells; CD19+ B-cells, CD19+CD43+CD93+CD23-CD21-age-associated B-cells; F4/80+siglec f+ eosinophils; F4/80HCD11clnt macrophages; F4/80IntCD11cH dendritic cells and LyG6+ neutrophils.

FIG. 5: PEPITEM treatment had no effect on metabolic changes associated with an obesogenic diet. Mice were fed HFD for either (A-B) 6 or (C-D) 12 weeks and received 0.0822 mg/week of PEPITEM or PBS as a control for either the full duration or for the last 6 weeks of the HFD. Body weight was assessed weekly for (A) 6 or (C) 12 weeks of HFD. Fasting blood glucose concentrations were measured at over 120 minutes in mice following (B) 6 or (D) 12 weeks of HFD. Data points from PBS treated mice are shown as black circles and data points from PEPITEM treated mice are shown as grey squares. Data are mean±SEM using (A-B) n=5 or (C-D) n=14 mice per group from n=1 independent experiment.

FIG. 6: Table showing quantification of other leukocyte subsets in mice on HFD for 6 weeks, where ABC=age-associated B-cells; ILN=inguinal lymph node; T reg=regulatory T cells; and data are shown as mean±SEM for n=1 independent experiments, using n=14 mice per group.

FIG. 7: Graphs showing the effects of PEPITEM on the production of TNFalpha (upper panel) and IL-6 (lower panel) from J774 monocyte/macrophage cells upon LPS stimulation.

### DETAILED DESCRIPTION OF THE INVENTION

The peptides of the invention, and the associated methods and uses, are surprisingly effective at reversing the effects of an obesogenic diet on pancreatic beta-cell size/area, aberrant T-cell trafficking into visceral adipose tissue (VAT) and leukocyte mobilisation from secondary lymphoid organs (SLOs). Consequently, the peptides of the invention are useful in methods of prophylaxis or treatment of obesity-associated inflammatory conditions and co-morbidities when administered to a patient in effective amounts. Such methods may be used to treat patients suffering from or prone to obesity and/or type 2 diabetes mellitus (T2DM). Therefore, the peptides of the invention are useful in the treatment and/or prophylaxis of obesity-associated inflammatory conditions such as obesity-induced chronic low-grade systemic inflammation and pancreatic beta-cell damage. The peptide, methods and uses of the invention are described in detail below.

In the discussion that follows, reference is made to a number of terms, which have the meanings provided below, unless a context indicates to the contrary. The nomenclature used herein for defining compounds, in particular the compounds according to the invention, is in general based on the rules of the IUPAC organisation for chemical compounds, specifically the “IUPAC Compendium of Chemical Terminology (Gold Book)”.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “treatment” defines the therapeutic treatment of a human or non-human animal patient, in order to impede or reduce or halt the rate of the progress of the condition, or to ameliorate or cure the condition. “Prophylaxis” of the condition as a result of treatment is also included. References to prophylaxis are intended herein not to require complete prevention of a condition: its development may instead be hindered through treatment in accordance with the invention. Typically, treatment is not prophylactic, and the compound or composition is administered to a patient having a diagnosed or suspected condition. By an “effective amount” herein defines an amount of the compound or composition of the invention that is sufficient to impede the noted diseases and thus produces the desired therapeutic or inhibitory effect.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superposable, i.e. cannot be brought into coincidence by translation and rigid rotation transformations. Enantiomers are chiral molecules, i.e. are distinguishable from their mirror image.

The term “racemic” is used herein to pertain to a racemate. A racemate defines a substantially equimolar mixture of a pair of enantiomers.

The term “diastereoisomers” (also known as diastereomers) defines stereoisomers that are not related as mirror images.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “prodrug” is used herein to refer to a compound which acts as a drug precursor and which, upon administration to a subject, undergoes conversion by metabolic or other chemical processes to yield a compound of formula (I).

The term “pharmaceutically acceptable excipient” defines substances other than a pharmacologically active drug or prodrug, which are included in a pharmaceutical product.

The term “intravenous administration” defines administration of a compound into a vein or veins.

The term “intramuscular administration” defines administration of a compound into a muscle.

The term “subcutaneous administration” defines administration of a compound into the subcutis, i.e. the layer of skin directly below the dermis and epidermis.

The term “oral administration” defines administration of a compound through the mouth, wherein the compound is typically in the form of a tablet or capsule.

The term “biocompatible” is used herein to refer to a material that is not harmful or toxic to living tissue.

The peptide of the invention is for use in the prophylaxis or treatment of obesity-associated inflammatory conditions. Obesity is defined herein to be an excessive body fat accumulation that presents a risk to health. A body mass index (BMI) of over 30 is defined by the World Health Organisation to be obese. Body fat consists of adipose tissue (AT), which is involved in numerous metabolic, hormonal and immunological processes. One of the symptoms of excessive AT is local and systemic inflammation, which is characterised by increased immune cells within AT. This may lead to co-morbidities including type 2 diabetes mellitus (T2DM), hepatic steatosis, cardiovascular disease, diabetic retinopathy and neurodegeneration (see, for example S. Winer et al., Nat. Med. 2009, 15 (8), 921-929), all of which are included within the definition of “obesity-associated inflammatory conditions”. Accordingly, in some embodiments, the peptide of the invention is for use in the prophylaxis or treatment of one or more selected from the group consisting of obesity-induced chronic low-grade systemic inflammation, type 2 diabetes mellitus, obesity-induced hepatic steatosis, obesity-induced cardiovascular disease, obesity-induced diabetic retinopathy and obesity-induced neurodegeneration.

Excessive AT also leads to dysregulation in metabolic processes involved in insulin sensitivity, leading to pancreatic beta-cell damage, insulin resistance and eventually hyperglycemia that underpins T2DM.

Obesity-associated inflammatory conditions are defined herein to be any inflammatory conditions caused by or linked to obesity, or any condition resulting from such an inflammatory condition.

In some embodiments, the obesity-associated inflammatory condition is at least one of obesity-induced chronic low-grade systemic inflammation and T2DM. Obesity-induced chronic low-grade systemic inflammation is characterised by increased proinflammatory cytokine secretion from AT and increased leukocytes within AT.

T2DM is progressive failure of pancreatic islet B-cells to compensate for insulin resistance and is characterized by high blood sugar, insulin resistance, and relative lack of insulin. Common symptoms include increased thirst, frequent urination, unexplained weight loss, increased hunger, feeling tired, and sores that do not heal. Long-term complications from high blood sugar include heart disease, strokes, diabetic retinopathy, kidney failure, and poor blood flow in the limbs.

Hepatic steatosis is also known as fatty liver disease and is a condition where excess fat builds up in the liver, resulting in tiredness and pain and sometimes more severe morbidities such as cirrhosis, liver cancer and esophageal varices. Hepatic steatosis is commonly associated with metabolic syndromes including obesity and T2DM.

Cardiovascular disease refers herein to a disease attributed to a blood clot or a build-up of fatty deposits inside a blood vessel such as an artery or vein. Cardiovascular disease includes coronary heart disease, stroke, and peripheral arterial disease. Cardiovascular disease is commonly associated with obesity and T2DM.

Diabetic retinopathy is a complication of diabetes, caused by high blood sugar levels damaging the blood vessels and/or nerve tissues at the back of the eye (retina), leading to reduced vision.

Neurodegeneration is the progressive loss of structure or function of neurons. A strong correlation lies between obesity and the development of Alzheimer's disease (AD) and Parkinson's disease (PD). Metabolic changes caused by obesity are related to damage to the central nervous system (CNS), which can lead to neural death, either by apoptosis or cell necrosis, as well as alter the synaptic plasticity of the neuron.

The peptide of the invention comprises the amino acid sequence SVTEQGAELSNEER, or variants thereof. Variants of the amino acid sequence are envisaged, provided that such variants are able to treat or prevent obesity-associated inflammatory conditions. Variants may have an improved ability to treat or prevent obesity-associated inflammatory conditions. This may be through changes in affinity for cognate receptor(s) or changes that alter the pharmacokinetic profile of the peptide in vivo. It will be appreciated that the person skilled in the art is capable of modifying peptides to increase their pharmacological ‘profile’ in vivo, and is aware that these changes are not based solely on amino acid substitution. Variants include peptides comprising a version of the amino acid sequence SVTEQGAELSNEER in which one or more amino acids, for example 1, 2, 3 or 4 amino acids, have been altered, either by deletion or substitution. Alternatively, the amino acid sequence may be altered by the addition of one or more amino acids, for example 1 to 6 amino acids.

In some embodiments, the variants comprise the amino acid sequence altered by substitution of one or more amino acids for another. The substitution may be a conservative replacement, by which is meant that any given amino acid is replaced by a different amino acid with similar biochemical properties. For example, where the amino acid is a serine or threonine, it may be replaced with a different amino acid selected from the group consisting of serine, cysteine, selenocysteine, threonine and methionine. Where the amino acid is a valine, glycine, alanine or leucine, it may be replaced with a different amino acid selected from the group consisting of glycine, alanine, valine, leucine and isoleucine. Where the amino acid is arginine, it may be replaced with a different amino acid selected from the group consisting of histidine and lysine. Finally, where the amino acid is a glutamate, asparagine or a glutamine, it may be replaced with a different amino acid selected from the group consisting of aspartate, glutamate, asparagine and glutamine.

The preferred peptide is 14 amino acids long, although the peptide can also be as few as 13, 12, 11 or 10 amino acids or as many as 15, 16, 17 18, 19 or 20 amino acids. Where amino acids are added or removed, these are preferably to or from the N and/or C terminus of the peptide.

Where the amino acid sequence is situated at either end of the peptide, variants also comprise the amino acid sequence modified at the N- or C-terminus with a chemical moiety. In some embodiments, the N-terminus of the peptide is modified such that one of the proton atoms bound to the nitrogen atom of the amino moiety is replaced with any one of the group consisting of acetyl, propionyl, myristoyl, palmitoyl, ubiquityl, biotinyl, dansyl, 2,4-dinitrophenyl, fluorescein, 7-methoxycoumarin acetic acid, and palmitic acid. In some embodiments, the C-terminus of the peptide is modified such that the hydroxy group bound to the carbon atom of the carboxylic acid moiety is replaced with an amino group, thereby forming an amide. Other modifications to the chemical structure that protect the peptide from degradation or clearance in vivo are also preferred variants, for example, PEGylation which utilises a linker or spacer as is known in the art.

In some embodiments, the peptide comprises the amino acid sequence SVTEQGAELSNEER. In other embodiments, the peptide is PEPITEM or variants thereof, i.e. it consists of the amino acid sequence SVTEQGAELSNEER, or variants thereof. In some embodiments, the peptide consists essentially of the amino acid sequence SVTEQGAELSNEER. In further embodiments, the peptide is PEPITEM, i.e. the peptide consists of the amino acid sequence SVTEQGAELSNEER.

“Consists essentially of” is used herein to refer to amino acid sequences sharing the same sequence as 12 or more of the amino acids of SVTEQGAELSNEER. For example, any one or two of the amino acids of this sequence may be deleted or replaced with any one or two different amino acids. Where the amino acids are replaced with different amino acids, it will typically be a conservative replacement.

The prophylaxis or treatment of the invention may comprise administering an effective amount of the peptide to a patient. The patient may be any animal susceptible to obesity and/or type 2 diabetes mellitus. In some embodiments, the patient is any one of the group consisting of mammal, bird, reptile and amphibian. In other embodiments, the patient is a mammal. In some embodiments, the patient is any one selected from the group consisting of human, horse, dog, cattle, goat, sheep, pig, cat, bison, camel, llama and alpaca. In more specific embodiments, the patient is any one selected from the group consisting of human, horse, dog, cattle, goat, sheep, pig and cat, most often a human.

The peptide of the invention may be in the form of a pharmaceutically acceptable salt. The term “pharmaceutically acceptable salt” is intended to define organic and/or inorganic salts that are pharmaceutically useful. The peptide may be isolated from reaction mixtures as a pharmaceutically acceptable salt. Alternatively, the pharmaceutically acceptable salt may be prepared in situ during the final isolation and purification of the peptide by reaction with a suitable base such as a hydroxide, carbonate or bicarbonate of a pharmaceutically acceptable metal cation, or with ammonia or a primary, secondary or tertiary amine. Pharmaceutically acceptable salts include cations based on alkali metals or alkaline earth metals such as lithium, sodium, potassium, calcium, magnesium and aluminium salts and nontoxic quaternary ammonia and amine cations including ammonium, tetramethylammonium, tetraethylammonium, methylamine, dimethylamine, trimethylamine, triethylamine, diethylamine, and ethylamine. Other examples of organic amines useful for the formation of base addition salts include ethylenediamine, ethanolamine, diethanolamine, piperidine, and piperazine.

The pharmaceutically acceptable salt may also be prepared by treatment of the peptide with a suitable acid, for example, hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, citric acid, benzoic acid and ascorbic acid.

The skilled person is aware that all naturally occurring amino acids with chiral carbon centres are formed in the L-configuration (levorotatory), with the exception of glycine, which has no chiral carbon centre. Therefore, when prepared from naturally-occurring amino acids, the peptide of the invention exists in one enantiomeric form, in which all amino acids are in the L-configuration. However, unnatural amino acids with chiral carbon centres may exist in the D-configuration (dextrorotatory) or in mixtures of both the L- or D-configuration. Therefore, when prepared from any unnatural amino acids, the peptide of the invention may exist in different enantiomeric forms. All enantiomers, diastereoisomers and racemic mixtures, are included within the scope of the invention. Individual stereoisomers of the peptide of the invention, i.e., associated with less than 5%, preferably less than 2% and in particular less than 1% of the other stereoisomer, are included. Mixtures of stereoisomers in any proportion, for example a racemic mixture comprising substantially equal amounts of two enantiomers are also included within the invention.

Also included are solvates and isotopically-labelled peptides. Isotopically-labelled peptides are identical to the peptides recited herein, but for the fact that one or more atoms are replaced by an atom having an atomic mass or mass number different from the atomic mass or mass number predominantly found in nature. Examples of isotopes that can be incorporated into peptides of the invention include isotopes of hydrogen, carbon, nitrogen, oxygen and sulfur, such as ²H, ³H, ¹³C, ¹⁴C, ¹⁵N, ¹⁸O, ¹⁷O, and ³⁵S, respectively.

Prodrugs of the peptide are also within the scope of the invention. Upon administration to a subject, a prodrug undergoes conversion by metabolic or other chemical processes to yield the peptide of the invention.

All amorphous and crystalline forms of the peptide of the invention are included.

The peptide of the invention may be administered alone. In some embodiments, the peptide of the invention is administered as part of a pharmaceutical composition. Such a pharmaceutical composition comprises an effective amount of the peptide of the invention, in combination with one or more pharmaceutically acceptable excipients. The excipient may aid transport of the peptide of the invention to the site in the body where it is intended to act, for example by increasing the rate of dissolution of the compound into the blood stream or by increasing the stability of the compound in order to delay its release, in order to increase its efficiency and prevent damage to tender tissues. Alternatively, the excipient may be for identification purposes, or to make the compound more appealing to the patient, for example by improving its taste, smell and/or appearance. Typically, the excipient makes up the bulk of the pharmaceutical composition.

Excipients include diluents or fillers, binders, disintegrants, lubricants, colouring agents and preservatives. Diluents or fillers are inert ingredients that may affect the chemical and physical properties of the final composition. If the dosage of the peptide is small then more diluents will be required to produce a composition suitable for practical use. If the dosage of the peptide is high then fewer diluents will be required.

Binders add cohesiveness to powders in order to form granules, which may form a tablet. The binder must also allow the tablet to disintegrate upon ingestion so that the peptide dissolves. Disintegration of the composition after administration may be facilitated through the use of a disintegrant.

An extensive overview of pharmaceutically acceptable excipients is described in the Handbook of Pharmaceutical Excipients, 6th Edition; Editors R. C. Rowe, P. J. Sheskey and M. E. Quinn, The Pharmaceutical Press, London, American Pharmacists Association, Washington, 2009. Any suitable pharmaceutically acceptable excipient is within the scope of the invention.

The peptide or pharmaceutical composition comprising the peptide of the invention may be administered by intravenous, intramuscular, subcutaneous or oral administration, where the peptide or composition may be administered by injection or by implant. In some embodiments, the peptide or composition comprising the peptide is suitable for intravenous, intramuscular or subcutaneous administration. In more specific embodiments, the peptide or composition comprising the peptide is suitable for subcutaneous administration by injection or by implant. In yet more specific embodiments, the peptide or composition comprising the peptide is suitable for subcutaneous administration by implant.

Implants suitable for release of the peptide or pharmaceutical composition comprising the peptide of the invention include any implant capable of controllably releasing the peptide, for example osmotic pumps.

Pharmaceutical compositions comprising the peptide of the invention may be compressed into solid dosage units, such as tablets, or be processed into capsules or suppositories. Where, a pharmaceutical composition comprising the peptide is injected, it is typically prepared in the form of a solution, suspension or emulsion for such. Alternatively, a pharmaceutical composition comprising the peptide may be administered as a spray. Otherwise, pharmaceutical compositions comprising the peptide of the invention may be processed into implants or any other preparations for immediate and/or sustained release.

Typically, pharmaceutical compositions comprising the peptide of the invention are processed into a solution, suspension or emulsion for intravenous, intramuscular or subcutaneous intrathecal administration and/or administration by implant.

Combination therapies comprising administration of the peptide of the invention together with at least one other drug that is known or suspected of being active for the treatment or prophylaxis of obesity-associated inflammatory conditions are also included within the invention. The peptide and at least one other drug may be administered together, for example as part of a pharmaceutical composition comprising pharmaceutically acceptable excipients such as those described above. Alternatively, the peptide and at least one other drug may be administered separately. Administration may be as described above in connection with the peptide of the invention. The at least one other drug may be known or suspected of being active for the treatment or prophylaxis of obesity-induced chronic low-grade systemic inflammation and type 2 diabetes mellitus. For example, the at least one other drug may be one or more selected from the group consisting of Repaglinide, Nateglinide, Glipizide, Glimepiride, Glyburide, Saxagliptin, Sitagliptin, Linagliptin, Alogliptin, Rosiglitazone, Pioglitazone, Acarbose, Miglitol, Canagliflozin, Dapagliflozin, Empagliflozin, Colesevelam, Pramlintide, Dulaglutide, Exenatide, Liraglutide, Lixisenatide, Semaglutide and Ertugliflozin.

In a further aspect, there is provided use of a peptide comprising the amino acid sequence SVTEQGAELSNEER, or variants thereof for the prophylaxis or treatment of obesity-associated inflammatory conditions.

In yet a further aspect there is provided use of a peptide comprising the amino acid sequence SVTEQGAELSNEER, or variants thereof for the manufacture of a medicament for the prophylaxis or treatment of obesity-associated inflammatory conditions.

When the peptide of the invention is used for the manufacture of a medicament, such a medicament includes any substance used for medical treatment. For the avoidance of doubt, implants lie within the definition of a medicament.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention as described herein without departing from the scope of the invention as described. The present embodiments are therefore to be considered for descriptive purposes and are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The aspects and embodiments of the invention are further described in the following clauses:

1. A peptide comprising the amino acid sequence SVTEQGAELSNEER, or variants thereof, for use in the prophylaxis or treatment of obesity-associated inflammatory conditions.

2. The peptide for the use of clause 1 wherein the prophylaxis or treatment is at least one of obesity-induced chronic low-grade systemic inflammation and type 2 diabetes mellitus.

3. The peptide for the use of clause 1 or clause 2 wherein the prophylaxis or treatment comprises administering the peptide intravenously, intramuscularly, subcutaneously, or orally.

4. The peptide for the use of any one previous clause wherein the prophylaxis or treatment comprises administering the peptide intravenously, intramuscularly or subcutaneously.

5. The peptide for the use of any one previous clause wherein the prophylaxis or treatment comprises administering the peptide by implant or injection.

6. The peptide for the use of clause 5 wherein the prophylaxis or treatment comprises administering the peptide by implant.

7. The peptide for the use of clause 5 or clause 6 wherein the implant allows for controlled release of the peptide.

8. The peptide for the use of any one of clauses 5 to 7 wherein the implant comprises an osmotic pump.

9. The peptide for the use of any one previous clause wherein the prophylaxis or treatment comprises administering the peptide to a patient in need of prophylaxis and/or treatment of obesity-associated inflammatory conditions.

10. The peptide for the use of clause 9, wherein the patient is a mammal.

11. The peptide for the use of clause 9, wherein the patient is any one selected from the group consisting of human, horse, dog, cattle, sheep, pig and cat.

12. The peptide for the use of clause 9, wherein the patient is a human.

13. The peptide for the use of any one preceding clause wherein the peptide consists of the amino acid sequence SVTEQGAELSNEER, or variants thereof.

14. The peptide for the use of any of clauses 1 to 12 wherein the peptide consists essentially of the amino acid sequence SVTEQGAELSNEER.

15. The peptide for the use of any of clauses 1 to 12 wherein the peptide consists of the amino acid sequence SVTEQGAELSNEER.

16. A method of prophylaxis or treatment of obesity-associated inflammatory conditions, comprising administering an effective amount of a peptide comprising the amino acid sequence SVTEQGAELSNEER, or variants thereof, to a patient in need thereof.

17. The method of clause 16 wherein the prophylaxis or treatment is as defined in any one of clauses 2 to 12.

18. The method of clause 16 or clause 17 wherein the peptide is as defined in any one of clauses 13 to 16.

19. Use of a peptide comprising the amino acid sequence SVTEQGAELSNEER, or variants thereof for the prophylaxis or treatment of obesity-associated inflammatory conditions.

20. Use of a peptide comprising the amino acid sequence SVTEQGAELSNEER, or variants thereof for the manufacture of a medicament for the prophylaxis or treatment of obesity-associated inflammatory conditions.

21. The use of clause 19 or clause 20 wherein the prophylaxis or treatment is as defined in any one of clauses 2 to 12.

22. The use of clause 19 or clause 20 wherein the peptide is as defined in any one of clauses 13 to 16.

The following are presented as non-limiting examples of the invention.

### EXAMPLES

Obesity drives systemic low-grade inflammation, altering metabolic and immune processes, and leading to numerous co-morbidities, including T2DM. In the examples below, it is shown that the immunopeptide PEPITEM is surprisingly effective in reversing the effects of an obesogenic-diet on (i) pancreatic beta-cell size; (ii) aberrant T-cell trafficking into VAT; (iii) leukocyte mobilisation from secondary lymphoid organs (SLOs); and (iv) reduction of obesity associated inflammatory markers TNFα and IL-6 in macrophages.

### Methods

### Mouse Models

Eight-week-old, male, C57BI/6J wild type (WT) mice were purchased from Charles River and were maintained in a specific pathogen free facility, with free access to food. Environmental conditions were: 21±2° C., 55±10% relative humidity and a 12 hour light-dark cycle. Mice were fed high fat diet (HFD) containing 60% fat (cat no. D12492, Research Diets, INC) for up to 12 weeks. Alzet mini pumps 2006 (Charles River) containing 3.5 mg/ml of PEPITEM (SVTEQGAELSNEER-PEG (352)-Amide; Cambridge Research Biochemicals Limited; Cambridge, UK) or phosphate buffered saline (PBS) were implanted at baseline (prophylactic administration) or following 6 weeks of HFD (therapeutic administration), continuously releasing 0.822 mg of content/week. Body weight was assessed weekly. Intraperitoneal glucose tolerance tests (IPGTT) were assessed at 3, 6, 8, and 12 weeks in mmol/l using Contour XT glucometer as described in Nasteska, D. et al., JCI Insight 6, doi: 10.1172/jci.insight. 140288 (2021). Mice were culled cardiac with by puncture blood collected into ethylenediaminetetraacetic acid (EDTA) coated eppendorfs^{RTM}. The peritoneal cavity was lavaged with 5 mM EDTA, spleen and inguinal lymph nodes were isolated and stored in PBS. Gonadal visceral fat pads and abdominal subcutaneous fat was stored in RPMI 1640 media (Gibco) containing 2% BSA and pancreas was FFPE as described in Nasteska, D. et al (supra).

### Sample Processing for Flow Cytometry Analysis

All tissue samples were weighed prior to processing. Fat tissue was broken into pieces, incubated in an enzyme cocktail consisting of 200 μg/ml Collagenase P (Sigma), 800 μg/ml Collagenase Dispase, 100 μg/ml DNase (both Merck) diluted in Roswell Park Memorial Institute medium (RPMI) containing 2% fetal bovine serum (FBS) at 37° C. for 30 min before being passed through 70 UM filter. Samples were washed by centrifugation in RPMI containing 2% FBS at 400 g (g-force) for 10 minutes and the stromal vascular fraction re-suspended in red blood cell (RBC) lysis buffer for 10 minutes. Samples were then washed by centrifugation at 400 g and re-suspended in RPMI containing 2% FBS. The spleen and lymph node were crushed through a 40 μM filter. Spleen, lymph node and blood samples were all incubated in the RBC lysis buffer as described prior to re-suspension in magnetic-activated cell sorting (MACS) buffer. Peritoneal lavage fluid (PLF) were centrifuged at 400 g for 5 minutes and the cells were resuspended in MACS buffer.

All samples were blocked with Fc receptor (FcR) blocker (Miltenyi Biotec) prior to staining with the following of antibodies and Zombie Aqua (Biolegend) for 20 minutes at 4° C. prior to washing and fixation with 2% paraformaldehyde (PFA): anti-CD45.2 BV605 (clone 104; Biolegend), anti-CD3 PECy7 (clone 145-2c11), anti-CD4 eFluor450 (clone GK1.5), anti-CD8 PE-TexasRed ^{RTM} (clone 5H10), anti-CD44 FITC (clone IM7), anti-CD25 AF700 (clone PC61.5), anti-KLRG1 APC-eFluor780 (clone 2F1), anti-CD62L PE (clone MEL-14), anti-CD19-APC (clone 1D3), anti-CD45 APC-CY7 (clone 104), F4/80 FITC (clone BM8), anti-CD11c PE-Cy7 (clone N418), anti-Gp38 PE (clone 8.1.1; all from Thermofisher); anti-CD23 BV421 (clone B3B4), anti-CD93 BV650 (clone AA4.1), anti-CD43 PerCp-Cy5.5 (clone S7), anti-CD21/35 PE (clone 7G6), anti-Siglec F TexasRed^{RTM} (clone E50-2440), Ly6G APC (clone 1A8; all from BD). Compensation controls were generated using the cells isolated from the spleen. Immediately prior to analysis CountBright beads (Invitrogen) were added and samples were acquired using Fortessa-X20 and data analysed offline using FlowJo^{RTM} (V-10.2.6) (FIG. 4).

### Immunofluorescence Imaging

Formalin-fixed paraffin-embedded (FFPE) pancreas tissue sections 5 μm in depth were subjected to deparaffinisation using histoclear (Sigma) following by incubation in decreasing concentrations of ethanol prior to antigen retrieval using a 10 mM citrate buffer at pH 6 for 25 minutes (see Nasteska, D. et al (supra)). Slides were blocked using 0.1% triton X-100 in 2% bovine serum albumin (BSA) for 1 hour, prior to incubation at 4° C. overnight with anti-insulin (1:500, clone C27C9, Cell Signalling) and anti-glucagon (1:2000, clone K79bB10, Sigma) antibodies diluted in blocking solution. Following 3×10-minute washes in blocking solution, slides were incubated for 2 hours in the dark at room temperature with goat-anti-mouse IgG AF488 and goat-anti-rabbit IgG AF633 (both Thermofisher) diluted in blocking solution. Slides were washed as described and mounted using Vectrashield Mounting Media with DAPI and stored at 4° C.

Slides were imaged using Zeiss Axioscan 7 Slide Scanner at 20× magnification using the same acquisition parameters for each slide. Images were initially processed with ZEN Software (Black Edition v2.2) into digital arrays, which were subsequently analysed with ImageJ (1.53K). Insulin and glucagon were used to identify beta and alpha cells, respectively and 4′,6-diamidino-2-phenylindole (DAPI) to identify total nuclei (cell number). Average number of islets and islet area were determined and expressed as number or area/10,000 μm² of the whole pancreatic section area, as previously described by Slavin, B et al., Anat. Rec. (Hoboken), 293, 108-116, doi: 10.1002/ar.21019 (2010).

### In Vitro Cytokine Analysis

J774 murine macrophages cells were pre-treated with 10 ng ml⁻¹ PEPITEM (SVTEQGAELSNEER; Cambridge Research Biochemicals Limited; Cambridge, UK) or phosphate buffered saline (PBS—as control) for 15 minutes and then stimulated with LPS (10 μg ml⁻¹) for 24 h. Cell supernatants were collected and analysed for the protein expression of IL-6 and TNF-α by ELISA.

### Statistical Analysis

Data were analysed using GraphPad Prism and presented as mean±SEM for n independent experiments. Normality was assessed using Shapiro-Wilk test. Univariate analysis was performed using unpaired t-test. p<0.05 was deemed statistically significant.

### Results

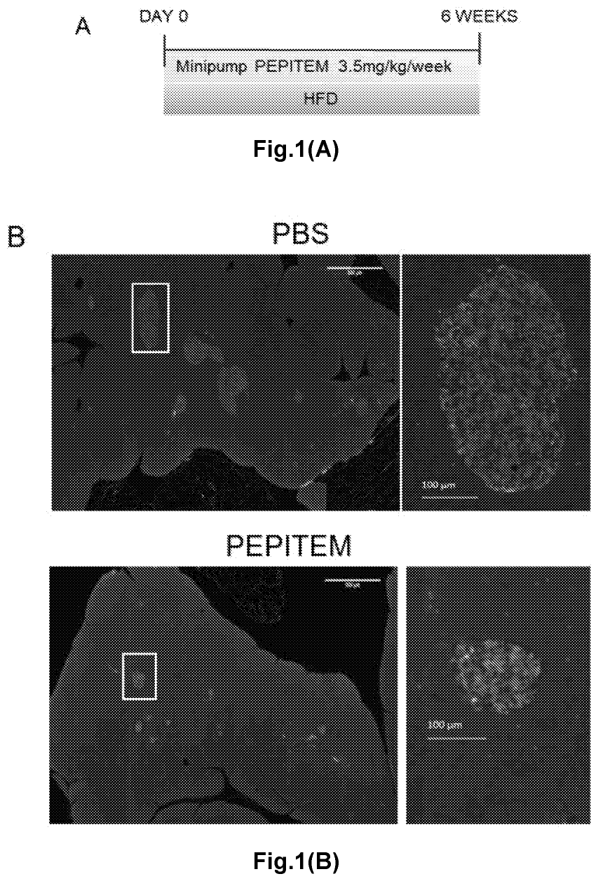

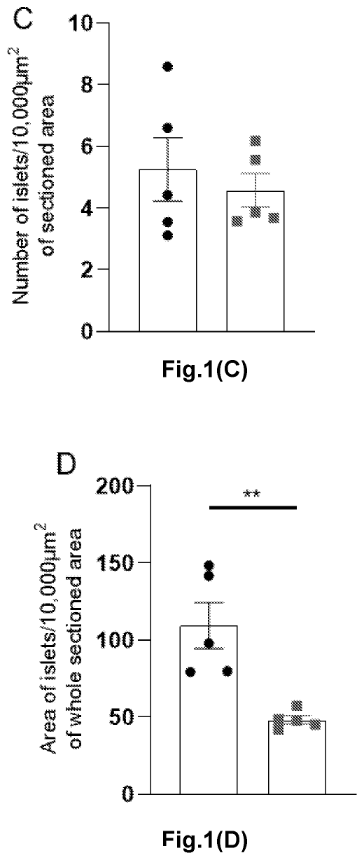

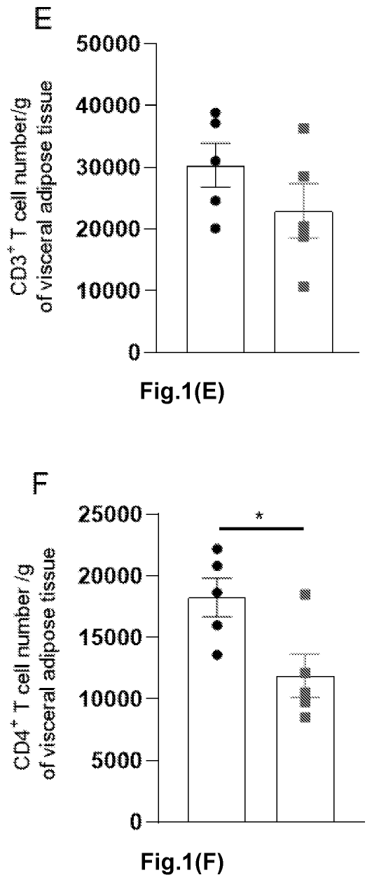

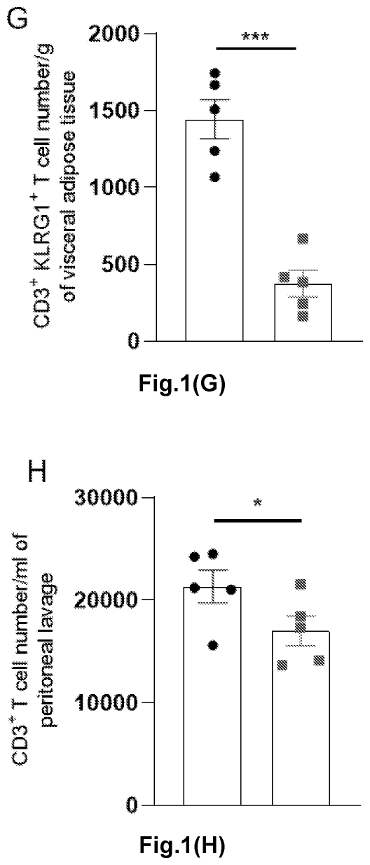

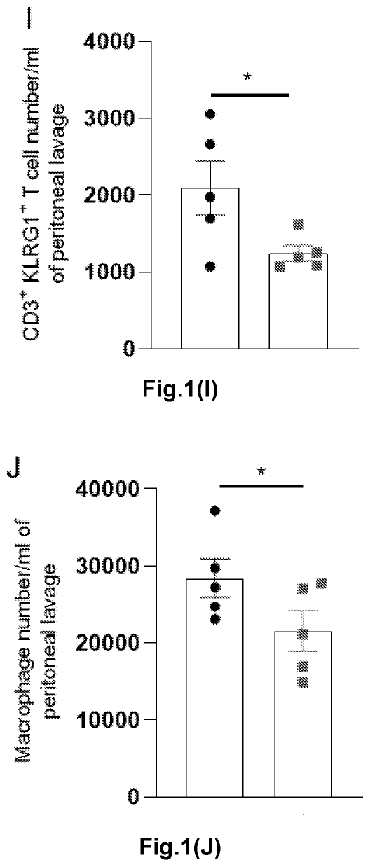

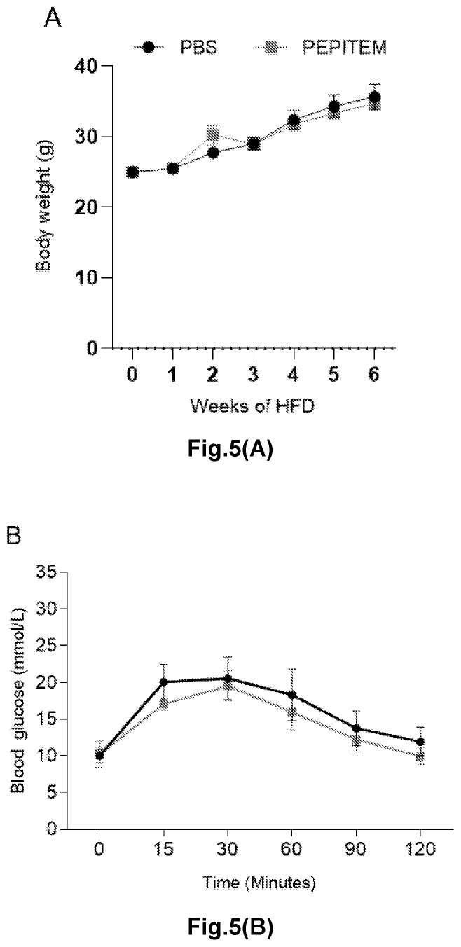

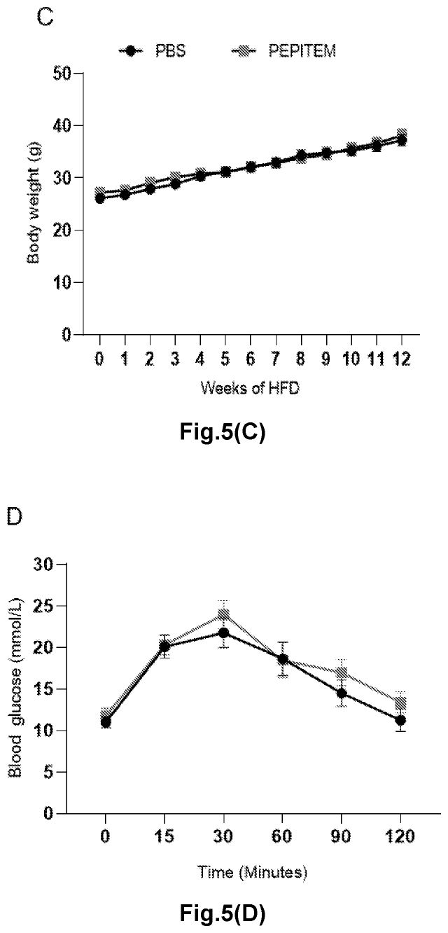

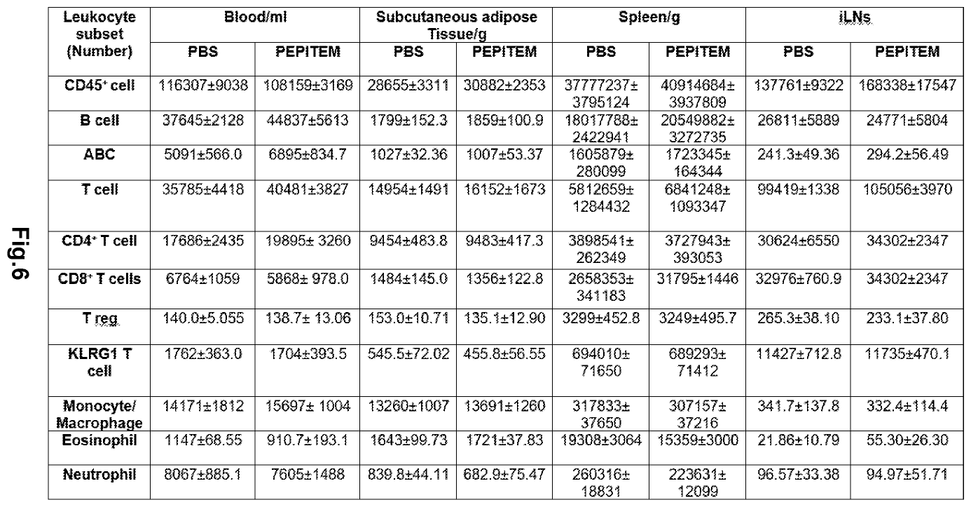

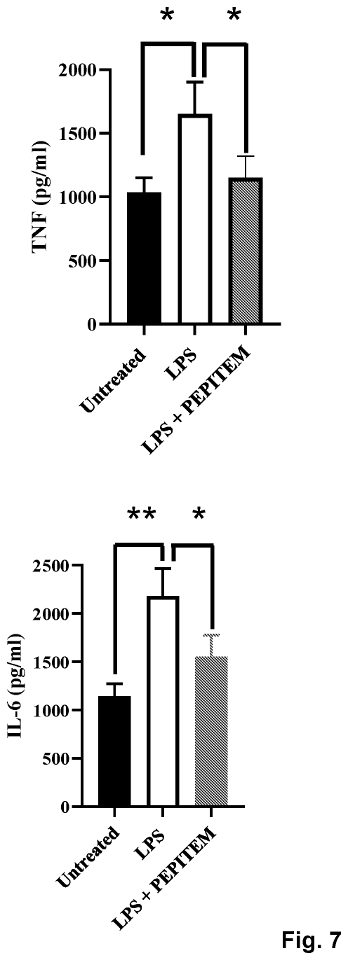

Obesity induces systemic metabolic and immunological changes driving various metabolic syndromes and immune-mediated inflammatory diseases (IMIDs). Initially the inventors examined whether prophylactic administration of the immunopeptide, PEPITEM, modulated obesogenic-diet inducing inflammation over a 6-week period (FIG. 1(A)). As expected body weight increased with the HFD over this timeframe and was unaffected by PEPITEM treatment (FIG. 5(A)). Similarly, PEPITEM had no effect on fasting glucose tolerance or insulin resistance at either week 3 or 6 of HFD (FIG. 5(B)). As pancreatic beta-cell mass is a key pathological feature in both obesity and T2DM (see Inaishi, J. & Saisho, Y., Nutrients, 12, doi: 10.3390/nu12123846 (2020)), the inventors assessed the effect of PEPITEM on the number and size of pancreatic islets following 6 weeks of HFD (FIG. 1(B)). Prophylactic PEPITEM treatment had no effect on the number of pancreatic islets (FIG. 1(C)), but significantly reduced their individual area when compared to PBS treated mice on HFD (FIG. 1(D)), indicating that PEPITEM was able to inhibit the dietary expansion of pancreatic islets. This is the first evidence that PEPITEM is having biological effects on non-immune cells.

Obesity and T2DM induce systemic low-grade chronic inflammation that is known to influence leukocyte composition, and thus their trafficking profiles, within peripheral tissues. Significantly fewer T-cells, in particular KLRG1+CD3+ T-cells, were found in the visceral adipose tissue (VAT) (FIGS. 1(E)-(G)) and within the peritoneal cavity (FIGS. 1(H)-(I)) in mice treated prophylactically with PEPITEM compared to the HFD control group. The inventors also observed a significant reduction of macrophages within the peritoneal cavity of PEPITEM treated animals compared to PBS (FIG. 1(J)) back to levels seen at in chow-fed 12-week-old mice (23,267±2,927 mean±SEM macrophages/ml, n=5). These data strongly indicated that PEPITEM is able to block the effects of an obesogenic-diet on T-cell and macrophage trafficking. Of note, prophylactic PEPITEM treatment had no effect on the number of T-cell subsets, B-cell subsets, neutrophils, eosinophils or macrophages in the blood, inguinal lymph node or spleen (FIG. 6).

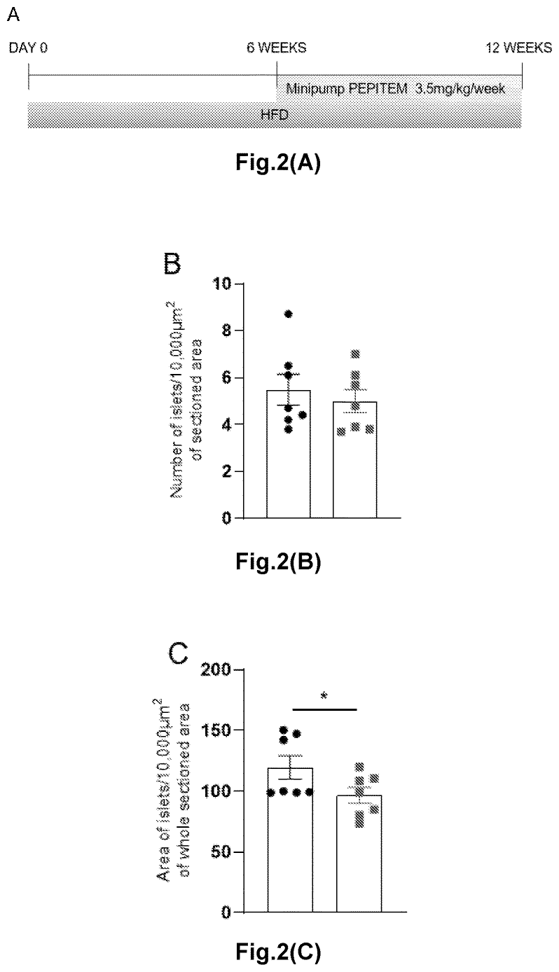

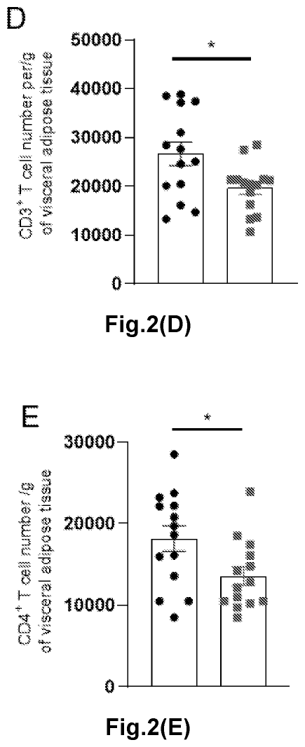

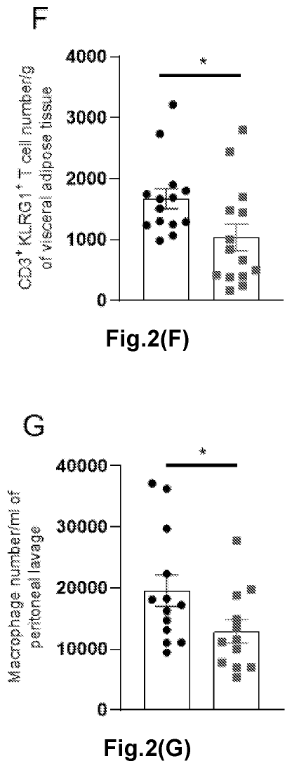

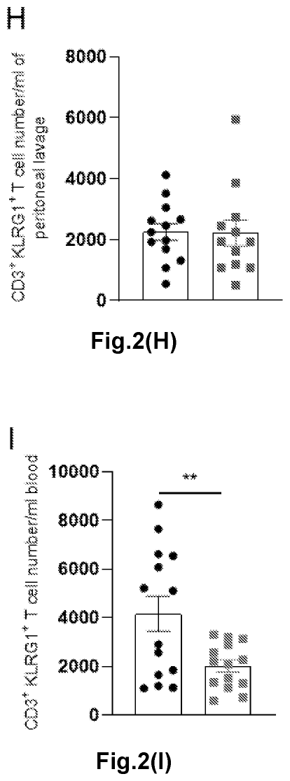

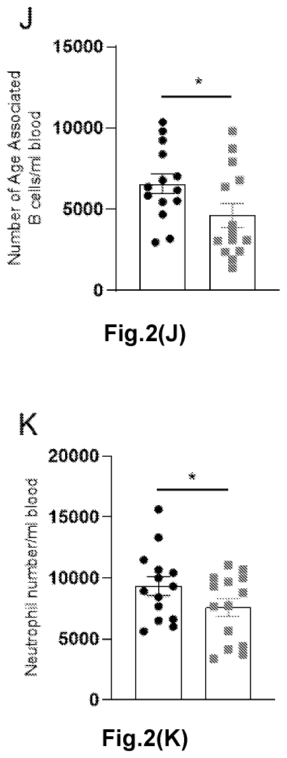

Next, we investigated the therapeutic potential of PEPITEM to reverse chronic inflammation and the development of T2DM/obese pancreas by feeding mice HFD prior to implantation of the PEPITEM slow-release pumps. As observed in the 6-week model, PEPITEM had no effect on the weight of the mouse or glucose tolerance at either 8 or 12 weeks of diet (FIG. 5(C)-(D)). However, therapeutic administration of PEPITEM (FIG. 2(A)) significantly reduced the pancreatic islet size, without affecting overall number of islets, when compared to control mice (FIG. 2(B)-(C)). This indicates that PEPITEM therapy can overcome/reverse diet induced increase in beta-cell area.

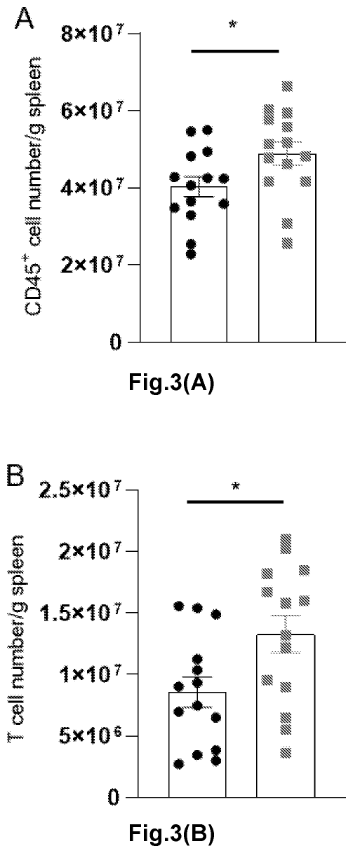

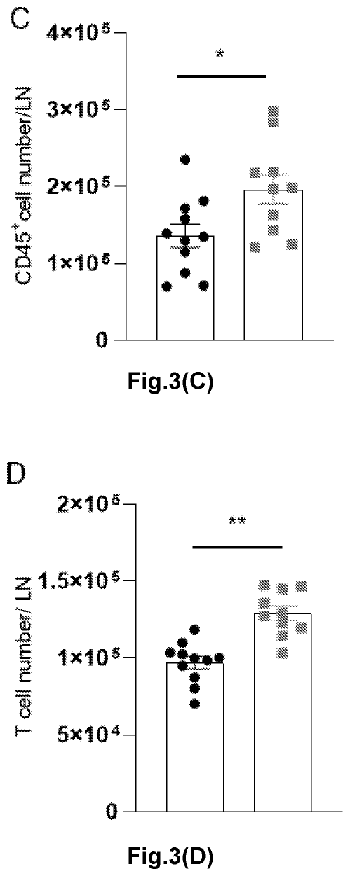

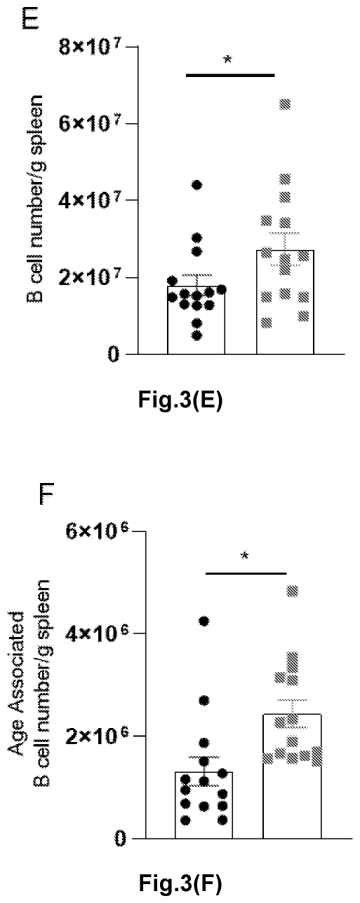

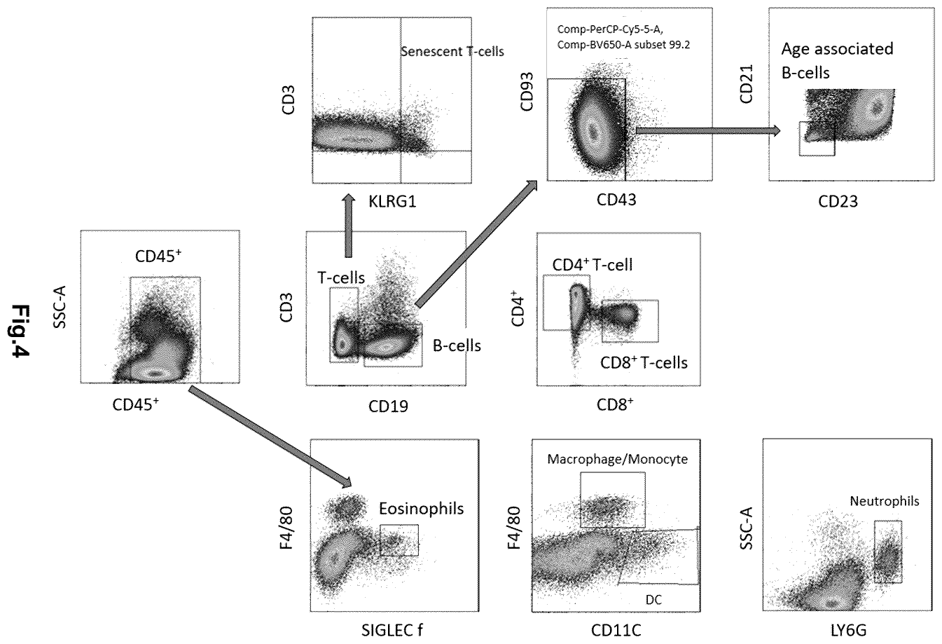

PEPITEM dramatically altered the composition of leukocyte subsets in the blood, inguinal lymph node, spleen, VAT and peritoneal cavity (FIG. 2(D)-(K)), but had no effect on subcutaneous adipose tissue (data not shown). Similar to the 6-week HFD model, the inventors observed a significant reduction in the number of CD3+ T-cells (FIG. 2(D)), CD4+ T-cells (FIG. 2(E)), CD3+KLRG1+ T-cells (FIG. 2(F)) in the VAT and in macrophage numbers in the peritoneal cavity (FIG. 2(G)) at 12 weeks of HFD following PEPITEM therapy compared to mice treated with vehicle control. Unlike the prophylactic study, the inventors observed no effect of therapeutic PEPITEM on CD3+KLRG1+ T-cells numbers in the peritoneal cavity (FIG. 2(H)) following 12 weeks of HFD. The number of CD3+KLRG1+ T-cells (FIG. 2(I)), age-associated B-cells (FIG. 2(J)), and neutrophils (FIG. 2(K)) were also lower in the blood of PEPITEM treated mice. By contrast, PEPITEM therapy increased the number of CD45+ leukocytes, in particular CD3+ T-cells, in the spleen (FIGS. 3(A)-(B)) and inguinal lymph node (FIGS. 3(C)-(D)) compared to control animals. B-cell (FIG. 3(E)) and age-associated B-cell (FIG. 3(F)) numbers were also found to be elevated in the spleen of PEPITEM treated mice compared to PBS treated mice. Thus, PEPITEM therapy systemically modulates leukocyte trafficking through the VAT, peritoneal cavity and secondary lymphoid organs (SLO), potentially reversing the pathologic effects of the obesogenic diet to these tissues.

Monocytes and/or macrophages play a fundamental role in maintaining adipose tissue homeostasis in health. The accumulation of pro-inflammatory macrophages (so called M1 macrophages) is widely accepted to play a central role in obesity-induced pathology. Next we investigated the direct effect of PEPITEM on monocyte/macrophage function, specifically their ability to release pro-inflammatory mediators (TNFalpha and IL-6) in response to an inflammatory stimuli (e.g., LPS). Here we see that PEPITEM significantly dampens the pro-inflammatory response of LPS-stimulated monocyte-macrophage cells (J774 cell line), resulting in a significant reduction in the amount of TNFalpha and IL-6 secreted. Therefore in addition to regulating cell migration during obesity, PEPITEM is able to regulate the effector function of monocyte/macrophages to limit the obesity-driven inflammatory response.

### DISCUSSION

As described above, 14-3-3ζ family members have been postulated to influence pancreatic beta cell proliferation; significantly higher levels of proliferation have been observed in vitro when murine or human islet beta cell were cultured in the presence of a pan 14-3-3 inhibitor (see Mugabo, Y. et al., supra.). In beta-cell specific 14-3-3ζ (parent protein of PEPITEM) KO mice, a significant increase in pancreatic beta cells proliferation was detected but this did not alter islet mass or area (see Mugabo, Y. et al., JCI Insight 7, doi: 10.1172/jci.insight. 156378 (2022)).

Unexpectedly, the inventors have found that PEPITEM treatment, either prophylactically or therapeutically, alleviated the effects of HFD on the pancreas, reducing pancreatic beta cell size. PEPITEM had no effect on obesogenic diet induced weight gain and therefore lipid storage in adipose tissues. This is the first evidence that PEPITEM can modulate the function of non-immune cells. Collectively, these studies indicate the potential for certain clinical interventions to uncouple obesity-induced changes in pancreatic homeostasis from those associated with lipid metabolism/storage in the adipose tissue and weight gain-thus offering an alternative means to reduce the risk of developing T2DM associated pancreatic damage in individuals at high risk.

Additionally, obesity has been reported to alter the trafficking profiles of T-cells, where memory CD4+ T-cells preferentially migrate to non-lymphoid (i.e. peripheral tissues) in a CXCR3 dependent manner. Elevated numbers of senescent or senescent-associated (CD153+) T-cells within the VAT of obese mice have also been described and senescent associated CD153+CD4+ T-cells from obese mice have been reported to drive metabolic dysregulation and inflammation within the VAT. Likewise, greater numbers of senescent CD8+CD57+ T-cells were detected in omental tissues of pre-diabetic or T2DM patients.

The inventors report for the first time that PEPITEM treatment, either prophylactically or therapeutically, limits T-cell trafficking (CD4+ T-cells, CD3+KLRG1+ senescent T-cells) to obese VAT. Crucially, levels of CD3+KLRG1+ senescent T-cells are also reduced in obese VAT following either prophylactic or therapeutic treatment, indicating that their production and/or trafficking is influenced by PEPITEM. These results suggest that PEPITEM is able to reverse the metabolic effects of HFD, and restore normal trafficking patterns between peripheral and lymphoid tissues.

There are a few studies describing alterations in leukocyte numbers within secondary lymphoid organs (SLOs) in preclinical models of obesity, which in some cases have been attributed to pathological changes in the barrier function of the lymphatics and abnormal lymph node architecture. For example, T-cell migration into the mesenteric lymph node and dendritic cell migration into local draining lymph nodes were reduced in obese subjects and loss of CCL21 gradients resulted in aberrant lymph node follicle organisation in obese mice, leading to a reduction in CD4+ and CD8+ T-cell numbers within the SLO. The inventors' data suggests that PEPITEM therapy potentially reverses these pathogenic effects, as elevated numbers of T and B-cells were observed in the SLO analysed here when compared to the untreated HFD controls. These changes were not observed following prophylactic PEPITEM administration over 6 weeks.

Numerous studies have reported influx of monocytes into VAT at the early stages of HFD-induced obesity and T2DM1, where they differentiate into M1 macrophages to drive pro-inflammatory responses. Whilst PEPITEM did not influence the absolute numbers of macrophages in the VAT, treatment with PEPITEM did alter macrophage numbers in the peritoneal cavity at both 6 and 12 weeks, where fewer macrophages were observed. Moreover, PEPITEM was able to dampen the effector function of macrophages, reducing the secretion of the pro-inflammatory cytokines, TNFα and IL-6.

As an endogenous molecule (akin to insulin), therapeutic administration of PEPITEM may provide the opportunity to re-establish control over both local and systemic metabolic and inflammatory processes underlying pathogenesis of obesity.

To conclude, therapeutic administration of the immunopeptide, PEPITEM, limits the pathological impact of obesity on the pancreas and systemic leukocyte trafficking, dampening the effects of obesity-induced systemic low-grade inflammation. Importantly, the inventors reveal for the first time that the actions of PEPITEM uncouple obesity-induced pathogenic lipid storage and metabolism in the adipose tissue (weight gain) from the systemic effects of obesity on pancreas homeostasis. Collectively, the data presented herein highlights the potential for PEPITEM as a novel therapy to combat the systemic low-grade inflammation experienced in obesity and minimise the impact of obesity on pancreatic homeostasis. Thus, offering an alternative strategy to reduce the risk of developing obesity-related co-morbidities, such as T2DM, in individuals at high risk and struggling to control their weight through life-style modifications.

1.-18. (canceled)

19. A method of prophylaxis or treatment of obesity-associated inflammatory conditions in a subject, the method comprising administering a peptide comprising the amino acid sequence SVTEQGAELSNEER, or variants thereof, to a subject in need thereof.

20. The method according to claim 19 wherein the prophylaxis or treatment is at least one of obesity-induced chronic low-grade systemic inflammation and type 2 diabetes mellitus.

21. The method according to claim 19 wherein the prophylaxis or treatment comprises administering the peptide intravenously, intramuscularly, subcutaneously, or orally.

22. The method according to claim 19 wherein the prophylaxis or treatment comprises administering the peptide intravenously, intramuscularly or subcutaneously.

23. The method according to claim 19 wherein the prophylaxis or treatment comprises administering the peptide by implant or injection.

24. The method according to claim 23 wherein the prophylaxis or treatment comprises administering the peptide by implant.

25. The method according to claim 23 wherein the implant allows for controlled release of the peptide.

26. The method according to claim 23 wherein the implant comprises an osmotic pump.

27. The method according to claim 19 wherein the prophylaxis or treatment comprises administering the peptide to a patient in need of prophylaxis and/or treatment of obesity-associated inflammatory conditions.

28. The method according to claim 27, wherein the patient is a mammal.

29. The method according to claim 27, wherein the patient is any one selected from the group consisting of human, horse, dog, cattle, sheep, pig and cat.

30. The method according to claim 27, wherein the patient is a human.

31. The method according to claim 19 wherein the peptide consists of the amino acid sequence SVTEQGAELSNEER, or variants thereof.

32. The method according to claim 19 wherein the peptide consists essentially of the amino acid sequence SVTEQGAELSNEER.

33. The method according to claim 19 wherein the peptide consists of the amino acid sequence SVTEQGAELSNEER.

34. A method of prophylaxis or treatment of obesity-associated inflammatory conditions, comprising administering an effective amount of a peptide comprising the amino acid sequence SVTEQGAELSNEER, or variants thereof, to a patient in need thereof.

35. The method according to claim 34 wherein the peptide consists essentially of the amino acid sequence SVTEQGAELSNEER.

36. The method according to claim 34 wherein the peptide consists of the amino acid sequence SVTEQGAELSNEER.


SEQUENCE LISTING (1 sequence)

SEQ ID NO: 1 - protein, 14 aa, synthetic construct
SVTEQGAELSNEER